(19) United States
(12) Patent Application Publication
(10) Pub. No.: US 2026/0200822 A1
(43) Pub. Date: Jul. 16, 2026

(54) PROCESS FOR PREPARING ALPHA,BETA-UNSATURATED CARBONYL COMPOUND

(71) Applicant: Firmenich SA, Satigny (CH)

(72) Inventors: Lucia BONOMO, Satigny (CH); Denis JACOBY, Satigny (CH); Fabien FONTENY, Satigny (CH); Fabrice KELLER, Satigny (CH)

(21) Appl. No.: 19/137,603

(22) PCT Filed: Dec. 15, 2023
(86) PCT No.: PCT/EP2023/086004

§ 371 Date: Jun. 10, 2025

(30) Foreign Application Priority Data
Dec. 15, 2022 (EP) 22213704.4

(51) Int. Cl. C07C 45/28; B01J 31/22

(52) U.S. Cl. CPC C07C 45/28; B01J 31/2208; B01J 31/2213; B01J 2231/70; B01J 2531/004; B01J 2531/0213; B01J 2531/0238; B01J 2531/26; B01J 2531/48

(57) ABSTRACT

Disclosed herein is a process for the oxidation of an allylic alcohol into an alpha,beta-unsaturated carbonyl compound in a presence of zirconium catalyst and a hydrogen acceptor.

### TECHNICAL FIELD

The present invention relates to the field of organic synthesis and more specifically it concerns a process for the oxidation of an allylic alcohol into an alpha,beta-unsaturated carbonyl compound in a presence of zirconium catalyst and a hydrogen acceptor.

### BACKGROUND

Alpha,beta-unsaturated carbonyl compounds represent skeletons highly desirables which could be used as such or as key intermediates useful to prepare more complex compounds in different fields such as, among others, perfumery, cosmetic, pharmaceutic or agrochemistry. In particular, carvone or (E)-4-methyldec-3-en-5-one are valuable compounds known as perfuming ingredient or could be a key intermediate toward more complex compounds. One access toward alpha,beta-unsaturated carbonyl compound is the oxidation of the corresponding alcohol, in particular Oppenauer oxidation which could be easily implemented at industrial scale. Nevertheless, such oxidation is carried out at high temperature. Moreover, as the Oppenauer reaction is reversible the hydrogen acceptor is added in increments and, then, the alcohol formed and non-reacted hydrogen acceptor are removed after each increment. However, today there is a need to develop sustainable process while reducing the energy consumption and simplifying the implementation of the process.

The present invention allows the oxidation of an allylic alcohol into an alpha,beta-unsaturated carbonyl compound under catalytic conditions and at a lower temperature while affording complete conversion and maintaining a high yield. To the best of our knowledge, the use of zirconium catalyst in such oxidation process has never been reported in the prior art.

### SUMMARY OF THE INVENTION

The invention relates to a novel process allowing the preparation of alpha, beta-unsaturated carbonyl compound under mild conditions never reported or suggested in the prior art allowing suppressing the addition of the portion wise hydrogen acceptor addition and several distillations along the process.

So, a first object of the present invention is a process for the oxidation of an allylic alcohol into an alpha, beta-unsaturated carbonyl compound wherein said process is carried out in the presence of
i) a catalyst of formula

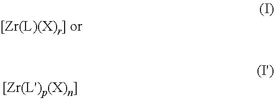

wherein L is a bisphenolate, a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units; and
ii) a hydrogen acceptor.

A second object of the present invention is a catalytic system comprising or consisting of
i) a catalyst of formula

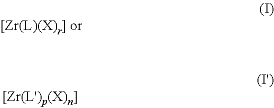

wherein L is a bisphenolate, a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units; and
ii) a hydrogen acceptor.

### DESCRIPTION OF THE INVENTION

Surprisingly, it has now been discovered that oxidation of an allylic alcohol into an alpha,beta-unsaturated carbonyl compound can be performed in an advantageous manner by means of catalyst of formula (I) or (I′) and a hydrogen acceptor. These unprecedented conditions allows the generation of an alpha,beta-unsaturated carbonyl compound in very high yield without requesting a high temperature, the portion wise addition of the hydrogen acceptor and the removal of alcohol formed by distillation before each addition of hydrogen acceptor. The inventions process provide a simple access to alpha,beta-unsaturated carbonyl compound.

Therefore, a first object of the present invention is a process for the oxidation of an allylic alcohol into an alpha,beta-unsaturated carbonyl compound wherein said process is carried out in the presence of
iii) a catalyst of formula

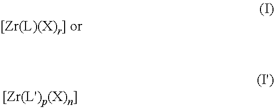

wherein L is a bisphenolate, a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units; and
iv) a hydrogen acceptor.

The term “alpha,beta-unsaturated carbonyl compound” is understood as an enal or an enone compound.

According to any embodiment of the invention, p is 1 and n is 3. In other words the catalyst of formula (I′) is of formula

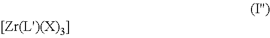

wherein L′ and X have the same meaning as defined above.

According to any embodiment of the invention, the phenolate is of formula

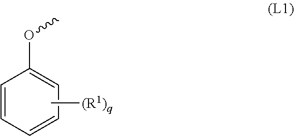

wherein the wavy line indicates the location of the bond between the Zirconium atom and L; q is an integer comprised between 0 and 3, R¹, simultaneously or independently, represents at least one substituent of the aromatic ring and is a halogen atom, a cyano group, a nitro group, a C₁₋₆ alkyl group optionally substituted with one or more halogen atoms, a C₁₋₆ alkoxy group or a COOR group wherein R is a hydrogen atom or a C₁₋₆ alkyl group.

The term “optionally” is understood that a group can or cannot comprise a certain functional group or substituent. The term “one or more” is understood as comprising 1 to 7, preferably 1 to 5 and more preferably 1 to 3 functional groups.

The term “alkyl group “alkenyl group” or “alkoxy group” are understood as comprising linear or branched alkyl, alkenyl or alkoxy groups. The term “alkanediyl group” or “alkenediyl group” are understood as comprising linear, branched, alicylic or cyclic alkanediyl or alkenediyl groups. The terms “alkenyl”; “alkenediyl” and “cycloalkenyl” are understood as comprising 1, 2 or 3 olefinic double bonds, preferably 1 or 2 olefinic double bonds, even more preferably 1 double bond. The term “cycloalkenyl” is understood as comprising a monocyclic or fused, spiro and/or bridged bicyclic or tricyclic cycloalkenyl group, preferably monocyclic cycloalkenyl group.

According to any embodiment of the invention, R¹ may be a halogen atom, a cyano group, a nitro group, a C₁₋₄ alkoxy group, a C₁₋₄ alkyl group optionally substituted with one to three halogen atoms or a COOR group wherein R is a hydrogen atom or a C₁₋₄ alkyl group. Particularly, R¹ may be a chlorine atom, a fluorine atom, a cyano group, a nitro group, a C₁₋₃ alkoxy group or a C₁₋₃ alkyl group optionally substituted with one to three halogen atoms or a COOR group wherein R is a hydrogen atom or a C₁₋₃ alkyl group. Particularly, R¹ may be a cyano group, a nitro group, a COOH group or a C₁₋₃ alkyl group optionally substituted with one to three fluorine atoms. Even more particularly, R¹ may be a cyano group, a nitro group or a methyl group optionally substituted with one to three fluorine atoms.

According to any embodiment of the invention, q may be 0 or 1, particularly 1.

According to any embodiment of the invention, R¹ may be, relative to position 1, an ortho substituent of the aromatic ring. In other words, the phenolate of formula (I) is of formula

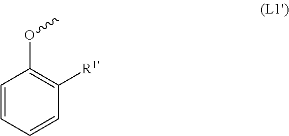

wherein the wavy line indicates the location of the bond between the Zirconium atom and L; R^{1′} is a hydrogen atom or a R¹ group as defined above

According to any embodiment of the invention, the phenolate is selected from the group consisting of 2-nitrophenolate, ortho-cresolate, 2-(trifluoromethyl)phenolate and 2-cyanophenolate.

According to any embodiment of the invention, the bisphenolate is of formula

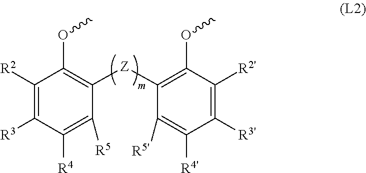

wherein the wavy lines indicate the location of the bond between the Zirconium atom and L; m is 0 or 1; R², R³ R⁴, R⁵, R^{2′}, R^{3′}, R^{4′} and R^{5′}, when taken separately, independently from each other, are a hydrogen, a halogen atom, a nitro group, a 2H-benzo[d][1,2,3]triazol-2-yl group, a C₁₋₆ alkoxy group, a C₁₋₆ thioalkyl group or a C₁₋₁₀ alkyl group optionally substituted with one or more of a halogen atom, hydroxy, amine or C₁₋₃ alkoxy group; or R² and R³ or R³ and R⁴ or R^{2′} and R^{3′} or R^{3′} and R^{4′}, when taken together represent a —O—(CH₂)_{y}—O— group wherein y is 1 or 2 or form a C₆ aryl, C₅₋₆ cycloalkyl group, each optionally substituted with one or more of a halogen atom, hydroxy or C₁₋₃ alkoxy group; and Z is an oxygen or sulphur atom, a (—CH₂—)₂ group, a —NH— group, a —SO₂— group, a —S—S— group, a —CH₂—NH—CH₂— group, or a —C(R⁶)(R⁷)— group wherein R⁶ and R⁷, when taken separately, independently from each other, are a hydrogen atom or a C₆ aryl, C₆ heteroaryl or a C₁₋₃ alkyl group, each optionally substituted by one to three halogen atoms or C₁₋₃ alkoxy groups, and wherein the heteroatom is one or more of an oxygen or nitrogen atom.

According to any embodiment of the invention, R², R³ R⁴, R⁵, R^{2′}, R^{3′}, R^{4′} and R^{5′}, when taken separately, independently from each other, may be a hydrogen atom, a chlorine atom or a C₁₋₉ alkyl group; or R² and R³ or R³ and R⁴ or R^{2′} and R^{3′} or R^{3′} and R^{4′}, when taken together form a C₆ aryl group.

According to any embodiment of the invention, R² and R^{2′}, independently from each other, may be a hydrogen atom or a C₁₋₄ alkyl group. Particularly, R² and R^{2′}, independently from each other, may be a hydrogen atom or a methyl or a tert-bulyl group. Particularly, R² and R^{2′} may be a hydrogen atom or a tert-bulyl group. Even more particularly, R² and R^{2′} may be a tert-bulyl group.

According to any embodiment of the invention, R³ and R^{3′}, independently from each other, may be a hydrogen atom or a C₁₋₄ alkyl group. Particularly, R³ and R^{3′}, independently from each other, may be a hydrogen atom or a C₁₋₃ alkyl group. Particularly, R³ and R^{3′}, independently from each other, may be a hydrogen atom or a C₁₋₂ alkyl group. Particularly, R³, independently from each other, may be a hydrogen atom or a methyl group. Even more particularly, R³ and R^{3′} may be hydrogen atom.

According to any embodiment of the invention, R⁴ and R^{4′}, independently from each other, may be a hydrogen atom or a C₁₋₉ alkyl group. Particularly, may be a hydrogen atom or a C₁₋₄ alkyl group. Particularly, R⁴ and R^{4′}, independently from each other, may be a hydrogen atom or a methyl or a tert-bulyl group. Even more particularly, R⁴ and R^{4′} may be a methyl or a tert-bulyl group.

According to any embodiment of the invention, R⁵ and R^{5′}, independently from each other, may be a hydrogen atom or a C₁₋₄ alkyl group. Particularly, R⁵ and R^{5′}, independently from each other, may be a hydrogen atom or a C₁₋₃ alkyl group. Particularly, R⁵ and R^{5′}, independently from each other, may be a hydrogen atom or a C₁₋₂ alkyl group. Particularly, R⁵, independently from each other, may be a hydrogen atom or a methyl group. Even more particularly, R⁵ and R^{5′} may be hydrogen atom.

According to any embodiment of the invention, R³ and R⁴, when taken together may form a C₆ aryl group.

According to any embodiment of the invention, R^{3′} and R^{4′}, when taken together may form a C₆ aryl group.

According to any embodiment of the invention, m is 1.

According to any embodiment of the invention, Z is a CR⁶R⁷ group.

According to any embodiment of the invention, R⁶ and R⁷, when taken separately, independently from each other, may be a hydrogen atom or a C₁₋₂ alkyl group. Particularly, R⁶ and R⁷, when taken separately, independently from each other, may be a hydrogen atom or a methyl group. Even more particularly, R⁶ may be hydrogen atom and R⁷ may be a hydrogen atom or a methyl group.

According to any embodiment of the invention, the bisphenolate may be selected from the group consisting of [1,1′-biphenyl]-2,2′-diol, [1,1′-binaphthalene]-2,2′-diol, 6,6′-methylenebis(2,4-di-tert-butylphenol), 6,6′-(ethane-1,1-diyl)bis(2,4-di-tert-butylphenol), 6,6′-methylenebis(2-(tert-butyl)-4-methylphenol), 6,6′-oxybis(2-(tert-butyl)-4-methylphenol) and 6,6′-thiobis(2-(tert-butyl)-4-methylphenol).

According to any embodiment of the invention, the triphenolate is of formula

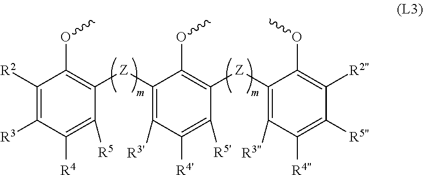

wherein the wavy lines indicate the location of the bond between the Zirconium atom and L; Z, m, R², R³ R⁴, R⁵, R^{2′}, R^{3′}, R^{4′} and R^{5′} have the same meaning as defined above and R^{2″}, R^{3″}, R^{4″} and R^{5″} is respectively a R², R³, R⁴, R⁵ group.

According to any embodiment of the invention, the triphenolate may be selected from the group consisting of 2,6-Bis[(2-hydroxyphenyl)methyl]phenol, 2,6-Bis[(2-hydroxy-3,5-dimethylphenyl)methyl]-4-methylphenol, 2,6-Bis(3-tert-butyl-5-methyl-2-hydroxybenzyl)-4-methylphenol, 4-Chloro-2,6-bis[(2-hydroxy-5-methylphenyl)methyl]phenol, 2,6-Bis[(5-chloro-2-hydroxyphenyl)methyl]-4-methylphenol, 2,6-Bis[(2-hydroxy-4-methylphenyl)methyl]-3-methylphenol, 4-Chloro-2,6-bis[(2-hydroxy-3,5-dimethylphenyl)methyl]phenol, 2,6-Bis[1-(2-hydroxyphenyl)ethyl]phenol, (2S)-1-[3,5-bis[[2,6-dihydroxy-4-methoxy-3-methyl-5-(1-oxobutyl)phenyl]methyl]-2,4,6-trihydroxyphenyl]-2-methyl-1-butanone, 2,2′-Methylenebis[6-[(2-hydroxy-5-methylphenyl)methyl]-4-methylphenol].

According to any embodiment of the invention, the calixarene may comprise 4, 6 or 8 phenol units, in particular 4 phenol units. The calixarene with 4 phenol units is of formula

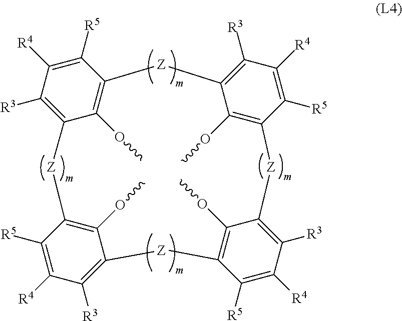

wherein the wavy lines indicate the location of the bond between the Zirconium atom and L; Z, m, R³, R⁴ and R⁵ have the same meaning as defined above

According to any embodiment of the invention, the calixarene with at least 4 phenol units may be selected from the group consisting of p-tert-Butylcalix[4]arene, 25,26,27,28-Tetrahydroxycalix[4]arene, Calix[6]arene, tert-Butylcalix[8]arene, tert-Butylcalix[6]arene, Calix[8]arene, p-Isopropylcalix[4]arene.

According to any embodiment of the invention, the anionic ligand, independently from each other, may be a halogen atom, a β-diketonate, a OOC⁸ group or a OR⁹ group wherein R⁸ is a C₁₋₁₀ alkyl group, a benzyl group, a naphtyl group or a phenyl group optionally substituted by a hydroxy group and R⁹ is a C₁₋₆ alkyl group. The term “β-diketonate” is understood as a ligand comprising a C(═O)—CH═C(O⁻) group. Particularly, the β-diketonate is of formula R¹⁰—C(═O)—CH═C(O⁻)—R¹¹ wherein R¹⁰ and R¹¹, independently from each other, are a C₁₋₆ alkyl group, particularly a C₁₋₄ alkyl group, even more particularly a methyl, propyl, isopropyl or a terbutyl group. Non-limiting example of β-diketonate may include 4-oxopent-2-en-2-olate, 2,2-dimethyl-5-oxohex-3-en-3-olate, 2,6-dimethyl-5-oxohept-3-en-3-olate or 2,2,6,6-tetramethyl-5-oxohept-3-en-3-olate. Particularly, R⁸ may be C₁₋₈ alkyl group, particularly a C₁₋₆ alkyl group, even more particularly a C₁₋₅ alkyl group. Particularly, R⁹ may be C₁₋₄ alkyl group, particularly a propyl group, a isoproly group, a butyl group or a tertbutyl group. Particularly, the anionic ligand is selected from the group consisting of acetylacetonate, acetate and pivalate.

According to a particular embodiment, when p is 0 and n is 4; X is a alkoxide of formula OR⁹ as define above.

According to any embodiment of the invention, the catalyst is of formula (I).

According to any embodiment of the invention, r is 2 and L is a bisphenolate. According to any embodiment of the invention, the catalyst of formula (I) may be selected from the group consisting of Zirconium, [2,2′-methylenebis[(4-methyl-6-tert-butyl)phenolato]]bis(2,4-pentanedionato); Zirconium, [2,2′-methylenebis[(4-6-di-tert-butyl)phenolato]]bis(2,4-pentanedionato); Zirconium, [2,2′-methylenebis[(4-6-di-methyl)phenolato]]bis(2,4-pentanedionato); Zirconium, [2,2′-ethylidenenebis[(4-6-di-tert-butyl)phenolato]]bis(2,4-pentanedionato); Zirconium, [2,2′-thiobis[(4-methyl-6-tert-butyl)phenolato]]bis(2,4-pentanedionato); Zirconium, [2,2′-oxobis[(4-methyl-6-tert-butyl)phenolato]]bis(2,4-pentanedionato); Zirconium, [[2,2′-binaphthalene]-1,1′-diolato]bis(2,4-pentanedionato); Zirconium, [[2,2′-binaphthalene]-1,1′-diolato]bis(propanolate); Zirconium, [[2,2′-biphenyl]-1,1′-diolato]bis(propanolate).

The catalyst of formula (I) or (I′) can be added into the reaction medium of the invention's process to form an alpha, beta-unsaturated carbonyl compound in a large range of concentrations. As non-limiting examples, one can cite, as catalyst concentration values those ranging from 0.1 mol % to 10 mol %, relative to the total amount of the allylic alcohol. Particularly, the catalyst concentration may be comprised between 0.5 mol % to 5 mol %. It goes without saying that the process works also with more catalyst. However the optimum concentration of catalyst will depend, as the person skilled in the art knows, on the nature of the latter, on the nature of the allylic alcohol, on the temperature and on the desired time of reaction.

The catalyst of formula (I) or (I′) is can be prepared by several methods starting for example from Zr(acac)₄ or Zr(OPr)₄. Alternatively, The catalyst of formula (I) or (I′) is formed in situ by the reaction between Zr(OPr)₄, a phenol or a bisphenol and acetylacetone.

According to any one of the above embodiments of the invention, hydrogen acceptor is a hydrocarbon comprising at least one carbonyl functional group and having a boiling point equal to or greater than 80° C., preferably equal to or greater than 110° C., even more preferably, equal to or greater than 120° C. Said hydrogen acceptor reacts with generated hydrogen and generates an alcohol. The hydrogen acceptor may be a aldehyde or a ketone. In particular, the borrowing hydrogen source may be of formula

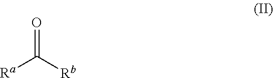

wherein R^{a} represents a C₁₋₁₀ linear alkyl group optionally substituted by a hydroxy group or an aryl group, a C₂₋₁₀ linear alkenyl group optionally substituted by a hydroxy group or an aryl group, a C₃₋₁₀ branched or cyclic alkyl or alkenyl group optionally substituted by a hydroxy group or an aryl group, or a phenyl group optionally substituted by one to five C₁₋₃ alkyl or alkoxy groups, hydroxy groups or halogen atoms; R^{b} represents a hydrogen atom or a R^{a} group; or R^{a} and R^{b}, when taken together, represent a C₂₋₁₀ linear or branched alkanediyl or alkenediyl optionally substituted by a hydroxy group or an aryl group. The hydrogen acceptor of formula (II) is a C₄₋₁₀ compound.

The terms “aryl group” or “heteroaryl group” designate the normal meaning in the art; i.e. an aromatic hydrocarbon group such as phenyl, pyridine or naphthyl group optionally substituted. Non-limiting examples of the optional substituent of the aryl group may include C₁₋₃ alkyl or alkoxy group, a hydroxy group or a halogen atom.

According to any one of the above embodiments, R^{a} may represent a phenyl group; a C₁₋₁₀ linear alkyl group or a C₃₋₁₀ branched or cyclic alkyl group, each optionally substituted by a hydroxy group. Preferably, R^{a} may represent a phenyl group, a C₁₋₁₀ linear alkyl group optionally substituted by a hydroxy group or a C₃₋₁₀ branched or cyclic alkyl group optionally substituted by a hydroxy group. Preferably, R^{a} may represent a C₃₋₈ linear or branched alkyl group optionally substituted by a hydroxy group. Even more preferably, R^{a} may represent a phenyl, methyl, ethyl, propyl, isopropyl, butyl, isobutyl, pentyl, hexyl, heptyl octyl group.

According to any one of the above embodiments, R may represent a hydrogen atom or a C₁₋₁₀ linear alkyl group optionally substituted by a hydroxy group or an aryl group or a C₃₋₁₀ branched or cyclic alkyl group optionally substituted by a hydroxy group or an aryl group. Preferably, R may represent a hydrogen atom, a methyl, an ethyl or a propyl group.

According to any one of the above embodiments, R^{a} and R^{b}, when taken together, may represent a C₄₋₈ linear, branched alkanediyl or alkenediyl optionally substituted by a hydroxy group. Preferably, R^{a} and R^{b}, when taken together, may represent a C₄₋₇ linear, branched alkanediyl or alkenediyl optionally substituted by a hydroxy group. Preferably, R^{a} and R^{b}, when taken together, may represent a C₄₋₇ linear alkanediyl. Even more preferably, R^{a} and R^{b}, when taken together, may represent a C₄₋₅ linear alkanediyl.

Non-limiting example of suitable hydrogen acceptor may include the compounds selected from the group consisting of benzaldehyde, cyclohexanone, 2-heptanone, 2-octanone, 2-pentanone, acetophenone, 4-methyl-2-pentanone, isophorone, 3-methyl-2-butanone, and a mixture thereof.

The hydrogen acceptor can be added into the reaction medium of the invention's process to form an alpha,beta-unsaturated carbonyl compound in a large range of concentrations. As non-limiting examples, one can cite, as hydrogen acceptor concentration values those ranging 1 equivalent to 5 equivalents, or even between 1 equivalent to 2 equivalents, relative to the amount of the allylic alcohol. It goes without saying that the process works also with more hydrogen acceptor. However the optimum concentration of hydrogen acceptor will depend, as the person skilled in the art knows, on the nature of the latter, on the nature of the allylic alcohol, on the temperature and on the desired time of reaction.

According to any one of the above embodiments, the allylic alcohol is a compound of formula

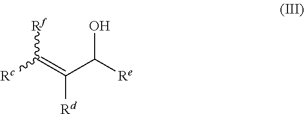

in a form of any one of its stereoisomers or a mixture thereof and wherein R^{c} is a hydrogen atom or a C₁₋₁₀ alkyl or a C₂₋₁₀ alkenyl group, R^{d} is a hydrogen atom or a C₁₋₃ alkyl group, R^{e} is a hydrogen atom or a C₁₋₅ alkyl group, R^{f} is a hydrogen atom or a methyl group; or R^{c} and R^{e} are taken together and represent a C₂₋₁₆ alkanediyl or a C₃₋₁₆ alkenediyl group; or R^{d} and R^{e} are taken together and represent a C₂₋₁₆ alkanediyl group; or R^{c} and R^{d} are taken together and represent a C₂₋₁₆ alkanediyl or a C₃₋₁₆ alkenediyl group; and the alpha,beta-unsaturated carbonyl compound is a compound of formula

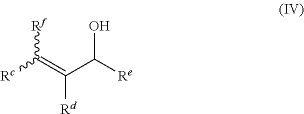

in a form of any one of its stereoisomers or a mixture thereof and wherein R^{c}, R^{d} and R^{e} have the same meaning as defined above.

According to any one of the above embodiments, R is a hydrogen atom.

According to any one of the above embodiments, the allylic alcohol is a compound of formula

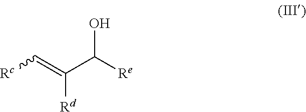

in a form of any one of its stereoisomers or a mixture thereof and wherein R^{e} is a hydrogen atom or a C₁₋₁₀ alkyl or a C₂₋₁₀ alkenyl group, R^{d} is a hydrogen atom or a C₁₋₃ alkyl group, R^{e} is a hydrogen atom or a C₁₋₅ alkyl group; or R^{c} and R^{e} are taken together and represent a C₂₋₁₆ alkanediyl or a C₃₋₁₆ alkenediyl group; or R^{d} and R^{e} are taken together and represent a C₂₋₁₆ alkanediyl group; or R^{e} and R^{d} are taken together and represent a C₂₋₁₆ alkanediyl or a C₃₋₁₆ alkenediyl group; and the alpha,beta-unsaturated carbonyl compound is a compound of formula

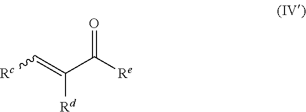

in a form of any one of its stereoisomers or a mixture thereof and wherein R^{c}, R^{d} and R^{e} have the same meaning as defined above.

According to any one of the above embodiments, the allylic alcohol is a compound of formula

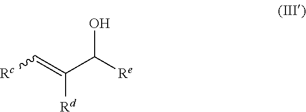

in a form of any one of its stereoisomers or a mixture thereof and wherein R^{e} is a a C₁₋₁₀ alkyl group, R^{d} is a hydrogen atom or a C₁₋₃ alkyl group, R^{e} is a C₁₋₃ alkyl group; or R^{c} and R^{e} are taken together and represent a C₂₋₁₆ alkanediyl group; or R^{d} and R^{e} are taken together and represent a C₂₋₁₆ alkanediyl group; or R^{c} and R^{d} are taken together and represent a C₂₋₁₆ alkanediyl group; and the alpha,beta-unsaturated carbonyl compound is a compound of formula

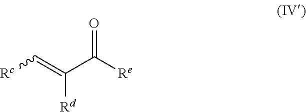

in a form of any one of its stereoisomers or a mixture thereof and wherein R^{c}, R^{d} and R^{e} have the same meaning as defined above.

The term “alkanediyl group” or “alkenediyl group” is understood as comprising linear, branched, cyclic or alicyclic alkanediyl or alkenediyl groups.

For the sake of clarity, by the expression “any one of its stereoisomers or a mixture thereof”, or the similar, it is meant the normal meaning understood by a person skilled in the art, i.e. that the compounds cited in the invention can be a pure enantiomer or a mixture of enantiomers. In other words, the compounds cited in the invention may possess at least one stereocenter which can have two different stereochemistries (e.g. R or S), e.g. the R^{c} group may comprise at least one stereocenter. Said compounds may even be in the form of a pure enantiomer or in the form of a mixture of enantiomers. The compounds cited in the invention may even be in the form of a pure diastereoisomer or in the form of a mixture of diastereoisomer when said compounds possess more than one stereocenter. Said compounds can be in a racemic form or scalemic form. Therefore, said compounds can be one stereoisomer or in the form of a composition of matter comprising, or consisting of, various stereoisomers.

The wavy line indicates that the double bond may be in the form of its E or Z isomer or of a mixture thereof; e.g. the invention comprises compositions of matter consisting of one or more compounds of formula (III) or one or more compounds of formula (IV), having the same chemical structure but differing by the configuration of the double bond.

According to any one of the above embodiments, R^{c} may be a hydrogen atom or a C₁₋₈ alkyl or a C₂₋₈ alkenyl group. Particularly, R^{c} may be a hydrogen atom or a C₁₋₆ alkyl or a C₂₋₆ alkenyl group. Particularly, R^{c} may be a hydrogen atom or a C₁₋₄ alkyl or C₄₋₆ alkenyl group. Particularly, R^{c} may be a hydrogen atom or a C₁₋₃ alkyl or C₄₋₆ alkenyl group. Particularly, R^{c} may be a hydrogen atom or a C₁₋₃ alkyl or C₄₋₆ alkenyl group. Particularly, R^{c} may be a hydrogen atom or a C₁₋₂ alkyl or C₆ alkenyl group. Even more particularly, R^{c} may be a hydrogen atom or a methyl, ethyl, hex-3en-1-yl or a 4-methylpent-3-en-1-yl.

According to any one of the above embodiments, R^{e} may be a hydrogen atom or a C₁₋₅ alkyl group. Particularly, R^{e} may be a hydrogen atom, a C₁₋₃ alkyl group or a pentyl group. Particularly, R^{e} may be a hydrogen atom or a methyl, ethyl or pentyl group. Particularly, R^{e} may be a hydrogen atom or a pentyl group. Even more particularly, R^{e} may be a hydrogen atom or a linear pentyl group.

According to any one of the above embodiments, R^{d} may be a hydrogen atom or a C₁₋₂ alkyl group. Particularly, R^{d} may be a hydrogen atom or a methyl group. Even more particularly, R^{d} may be a methyl group.

According to any one of the above embodiments, R^{c} and R^{d} are taken together and represent a C₂₋₁₂ alkanediyl or C₃₋₁₂ alkenediyl group. Particularly, R^{c} and R^{d} are taken together and represent a C₂₋₁₀ alkanediyl or C₃₋₁₀ alkenediyl group. Particularly, R^{c} and R^{d} are taken together and represent a C₂₋₈ alkanediyl or C₃₋ₛ alkenediyl group. Particularly, R^{c} and R^{d} are taken together and represent a C₄₋₈ alkanediyl or C₄₋₈ alkenediyl group. Particularly, R^{c} and R^{d} are taken together and represent a C₆₋₈ alkanediyl or C₆₋₈ alkenediyl group. Particularly, R^{c} and R^{d} are taken together and form a C₅₋₆ cycloalkenyl optionally substituted by a C₁₋₄ alkyl or alkenyl group. Even more particularly, R^{c} and R^{d} are taken together and represent a 2-isopropenyl-1,4-butanediyl or a group of formula (a)

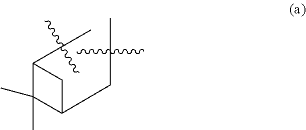 [SMILES: *CC1CC(*)C1(C)C]

in a form of any one of its stereoisomers or a mixture thereof.

According to any one of the above embodiments, R^{d} and R^{e} are taken together and represent a C₂₋₁₂ alkanediyl group. Particularly, R^{d} and R^{e} are taken together and represent a C₂₋₁₀ alkanediyl group. Particularly, R^{d} and R^{e} are taken together and represent a C₂₋₈ alkanediyl group. Particularly, R^{d} and R^{e} are taken together and represent a C₄₋₈ alkanediyl group. Particularly, R^{d} and R^{e} are taken together and represent a C₆₋₈ alkanediyl group. Even more particularly, R^{d} and R^{e} are taken together and represent a 2-a group of formula (a)

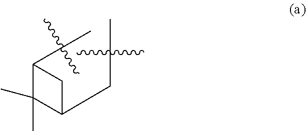 [SMILES: *CC1CC(*)C1(C)C]

in a form of any one of its stereoisomers or a mixture thereof.

According to any one of the above embodiments, R^{c} and R^{e} are taken together and represent a C₂₋₁₂ alkanediyl or C₃₋₁₂ alkenediyl group. Particularly, R^{c} and R^{e} are taken together and represent a C₂₋₁₀ alkanediyl or C₃₋₁₀ alkenediyl group. Particularly, R^{c} and R^{e} are taken together and represent a C₂₋₈ alkanediyl or C₃₋₈ alkenediyl group. Particularly, R^{c} and R^{e} are taken together and represent a C₄₋₈ alkanediyl or C₄₋₈ alkenediyl group. Particularly, R^{c} and R^{e} are taken together and represent a C₅₋₈ alkanediyl or C₅₋₈ alkenediyl group. Particularly, R^{c} and R^{e} are taken together and represent a C₅₋₇ alkanediyl or C₅₋₇ alkenediyl group. Particularly, R^{c} and R^{e} are taken together and represent a C₆ alkanediyl or C₆ alkenediyl group. Even more particularly, R^{c} and R^{e} are taken together and represent a 2-isopropenyl-1,3-propanediyl group, a group of formula (b) or a group of formula (c)

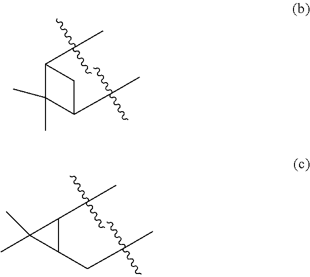

in a form of any one of their stereoisomers or a mixture thereof.

According to any one of the above embodiments, R^{c} and R^{e} are taken together and represent a C₂₋₁₂ alkanediyl group and R^{d} is a hydrogen atom or a C₁₋₃ alkyl group. Particularly, R^{c} and R^{e} are taken together and represent a C₂₋₈ alkanediyl group and R^{d} is a hydrogen atom or a C₁₋₃ alkyl group. Particularly, R^{c} and R^{e} are taken together and represent a C₂₋₆ alkanediyl group and R^{d} is a hydrogen atom or a C₁₋₃ alkyl group. Particularly, R^{c} and R^{e} are taken together and represent a C₂₋₄ alkanediyl group and R^{d} is a hydrogen atom or a C₁₋₃ alkyl group. Particularly, R^{c} and R^{e} are taken together and represent a C₂₋₄ alkanediyl group and R^{d} is a hydrogen atom or a C₁₋₂ alkyl group. Particularly, R^{c} and R^{e} are taken together and represent a C₂₋₃ alkanediyl group and R^{d} is a hydrogen atom or a C₁₋₂ alkyl group. Even more particularly, R^{c} and R^{e} are taken together and represent a C₃ alkanediyl group and R^{d} is a methyl group.

According to any one of the above embodiments of the invention, the allylic alcohol is carveol, (6,6-dimethylbicyclo[3.1.1]hept-2-en-2-yl)methanol, 4-methyl-3-decen-5-ol, 6,6-dimethyl-2-methylenebicyclo[3.1.1]heptan-3-ol i.e. pinocarveol, (4-(prop-1-en-2-yl)cyclohex-1-en-1-yl)methanol, 1-octen-3-ol, 4,7,7-trimethylbicyclo[4.1.0]hept-4-en-3-ol, 3,7,7-trimethylbicyclo[4.1.0]hept-3-en-2-ol geraniol, nerol, 4,6,6-trimethylbicyclo[3.1.1]hept-3-en-2-ol, (2E,6Z)-nona-2,6-dien-1-ol, 3-methyl-2-buten-1-ol, 6-isopropyl-3-methylcyclohex-2-en-1-ol, 5-isopropyl-2-methylcyclohex-2-en-1-ol, (6,6-dimethylbicyclo[3.1.1]hept-2-en-2-yl)methanol. Particularly, the allylic alcohol is carveol.

According to any one of the above embodiments of the invention, the alpha,beta-unsaturated carbonyl compound is carvone, 6,6-dimethylbicyclo[3.1.1]hept-2-ene-2-carbaldehyde, 4-methyldec-3-en-5-one, 6,6-dimethyl-2-methylidenebicyclo[3.1.1]heptan-3-one, 4-prop-1-en-2-ylcyclohexene-1-carbaldehyde, 1-octen-3-one, 4,7,7-trimethylbicyclo[4.1.0]hept-4-en-3-one, 3,7,7-trimethylbicyclo[4.1.0]hept-3-en-2-one, (2E)-3,7-dimethylocta-2,6-dienal, (2Z)-3,7-dimethylocta-2,6-dienal, 4,6,6-trimethylbicyclo[3.1.1]hept-3-en-2-one, (2E,6Z)-nona-2,6-dienal, 5-isopropyl-2-methylcyclohex-2-en-1-one, 6-isopropyl-3-methylcyclohex-2-en-1-one, or 3-methyl-2-butenal. Particularly, the alpha,beta-unsaturated carbonyl compound is carvone.

According to any one of the invention's embodiments, the invention's process for the oxidation of an allylic alcohol into an alpha,beta-unsaturated carbonyl compound is carried out at a temperature comprised between 50° C. and 190° C. In particular, the temperature is in the range between 100° C. and 140° C. Of course, a person skilled in the art is also able to select the preferred temperature as a function of the melting and boiling point of the starting and final products as well as the desired time of reaction or conversion.

The invention's process for the oxidation of an allylic alcohol into an alpha,beta-unsaturated carbonyl compound can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene or pseudocumene, or mixtures thereof, hydrocarbon solvents such as cyclohexane, heptane or mixtures thereof, nitrile solvent such as acetonitrile, esteral solvents such as ethyl acetate or ethereal solvents such as tetrahydrofuran, diethyether, methyl tetrahydrofuran or mixtures thereof. The choice of the solvent is function of the nature of the substrate and/or catalyst and the person skilled in the art is well able to select the solvent most suitable in each case to optimize the reaction.

The invention's process for the oxidation of an allylic alcohol into an alpha,beta-unsaturated carbonyl compound may carried out under batch or continuous conditions.

The invention's process for the oxidation of allylic alcohol into alpha,beta-unsaturated carbonyl compound may be performed under atmospheric pressure.

According to any one of the above embodiments, the allylic alcohol may be prepared by the rearrangement of an epoxide into an allylic alcohol. In other words, the invention's process comprises the step of
i) the rearrangement of an epoxide into an allylic alcohol; and
ii) the oxidation of the allylic alcohol obtained in step i) into alpha,beta-unsaturated carbonyl compound as defined above.

According to a particular embodiment of the invention, the rearrangement of the epoxide into allylic alcohol and the oxidation of the allylic alcohol into alpha,beta-unsaturated carbonyl compound can be performed in one-pot. The term “one-pot” is understood as both steps are performed successively in a single reaction system. In this case, the hydrogen acceptor and the zirconium catalyst are added after completion of the 1^{st} step. The Zirconium catalyst may also be generated in situ after completion of the 1^{st} step.

According to a particular embodiment of the invention, the rearrangement of the epoxide into allylic alcohol is carried out in the presence of a catalyst of formula Zn(aa)₂ wherein aa is an α-amino carboxylate having at least 3 carbon atoms or a β-amino carboxylate or a catalyst of formula Zn(carboxylate)₂; and an amino phenol.

According to any one of the above embodiments, the α-amino acid is selected from the group consisting of proline, valine, lysine, D-proline, D-valine, D-lysine L-proline, L-valine, L-lysine, 2-phenylglycine, phenyl alanine, 2-amino-2-methylpropanoic acid and a mixture thereof and the amino phenol is selected from the group consisting of 2-aminophenol, 2-aminomethylphenol and a mixture thereof.

According to any embodiments of the invention, the β-amino carboxylate is anthralinate.

According to any one of the above embodiments, the catalyst of formula Zn(carboxylate)₂ is selected from the group consisting of Zn(2-ethylhexenoate)₂, Zn(OAc)₂, Zn(benzoate)₂, Zn(Naphtenate)₂, Zn(laurate)₂, Zn(stearate)₂, Zn(palmitate)₂, Zn(2-octyldodecanoate)₂; and the amino phenol is selected from the group consisting of 2-aminophenol, 2-aminomethylphenol and a mixture thereof.

According to any one of the above embodiments, the epoxide is a compound of formula

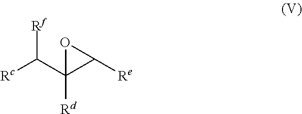

in a form of any one of its stereoisomers or a mixture thereof and wherein R^{c}, R^{d} and R^{e} have the same meaning as defined above.

According to any one of the above embodiments, the epoxide is a compound of formula

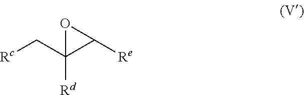

in a form of any one of its stereoisomers or a mixture thereof and wherein R^{c}, R^{d} and R^{e} have the same meaning as defined above.

According to any one of the above embodiments of the invention, non-limiting examples of the epoxide may be selected from the group consisting of 1,2-limonene oxide, 3,8,8-trimethyl-4-oxatricyclo[5.1.0.0^{3,5}]octane; 4,8,8-trimethyltricyclo[5.1.0.0^{2,4}]octane; 2-isopropyl-5-methyl-7-oxabicyclo[4.1.0]heptane; 4-isopropyl-1-methyl-7-oxabicyclo[4.1.0]heptane, alpha-pinene oxide, beta-pinene oxide, 2,2,6-trimethyl-1-oxaspiro[2.5]oct-5-ene; 2,2,6-trimethylspiro[2.5]oct-4-ene, caryophyllene oxide. Particularly, the epoxide may be 1,2-limonene oxide

The catalyst of formula Zn(aa)₂ or of formula Zn(carboxylate)₂ can be added into the reaction medium of the invention's process to form an allylic alcohol in a large range of concentrations. As non-limiting examples, one can cite, as catalyst concentration values those ranging from 0.1 mol % to 10 mol %, relative to the total amount of the epoxide. Particularly, the catalyst concentration may be comprised between 0.25 mol % to 5 mol %. It goes without saying that the process works also with more catalyst. However the optimum concentration of catalyst will depend, as the person skilled in the art knows, on the nature of the latter, on the nature of the epoxide, on the temperature and on the desired time of reaction.

According to any embodiments of the invention, the amino phenol is selected from the group consisting of 2-aminophenol, 2-aminomethylphenol, 2-Amino-4-chlorophenol, 2-Amino-3-methylphenol, 2-Amino-5-chlorophenol, 2-Amino-4-nitrophenol, 2-Amino-5-nitrophenol, 2-Amino-3-nitrophenol, 2-Amino-4-methoxyphenol, 2-Amino-4-tertbutylphenol and a mixture thereof.

The amino phenol can be added into the reaction medium of the invention's process to form an allylic alcohol in a large range of concentrations. As non-limiting examples, one can cite, as amino phenol concentration values those ranging 0.1 mol % to 20 mol %, or even between 0.5 mol % to 5 mol %, relative to the amount of the epoxide. It goes without saying that the process works also with more amino phenol. However, the optimum concentration of amino phenol will depend, as the person skilled in the art knows, on the nature of the latter, on the nature of the epoxide, on the nature of the catalyst, on the temperature and on the desired time of reaction.

According to any one of the invention's embodiments, the invention's process for the rearrangement of an epoxide into allylic alcohol is carried out at a temperature comprised between 50° C. and 190° C. In particular, the temperature is in the range between 100° C. and 190° C. In particular, the temperature is in the range between 100° C. and 185° C. In particular, the temperature is in the range between 160° C. and 190° C. Even more particularly, the temperature is in the range between 170° C. and 190° C. Of course, a person skilled in the art is also able to select the preferred temperature as a function of the melting and boiling point of the starting and final products as well as the desired time of reaction or conversion.

The invention's process for the rearrangement of an epoxide into allylic alcohol can be carried out in the presence or absence of a solvent. When a solvent is required or used for practical reasons, then any solvent current in such reaction type can be used for the purposes of the invention. Non-limiting examples include C₆₋₁₂ aromatic solvents such as xylene, toluene, 1,3-diisopropylbenzene, cumene or pseudocumene, or mixtures thereof, hydrocarbon solvents such as cyclohexane, heptane or mixtures thereof. The choice of the solvent is function of the nature of the substrate and/or catalyst and the person skilled in the art is well able to select the solvent most suitable in each case to optimize the reaction.

The invention's process for rearrangement of an epoxide into allylic alcohol may carried out under batch or continuous conditions.

The invention's process for the rearrangement of an epoxide into allylic alcohol may be performed under atmospheric pressure.

Another object of the present invention is a catalytic system comprising or consisting of
i) a catalyst of formula

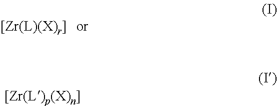

wherein L is a bisphenolate, a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units; and
ii) a hydrogen acceptor.

According to any embodiment of the invention, the catalytic system as defined above is suitable for use in the process for the oxidation of an allylic alcohol into an alpha,beta-unsaturated carbonyl compound as defined above.

Another object of the present invention is the use of a catalytic system comprising or consisting of
i) a catalyst of formula

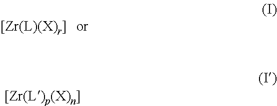

wherein L is a bisphenolate, a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units; and
ii) a hydrogen acceptor.
   in the process for the oxidation of an allylic alcohol into an alpha,beta-unsaturated carbonyl compound.

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). The preparation of precatalysts and ligands solutions were carried out under an inert atmosphere (Argon) using standard Schlenk techniques. The solvents were dried by conventional procedures and distilled under an argon atmosphere. NMR spectra were recorded at 20° C. on Bruker AV 300, AV 400, or AV 500 MHz spectrometers. Chemical shifts are reported in ppm relative to solvent signals (chloroform, δ_{H}=7.26 ppm, δ_{C}=77.0 ppm). The signal assignment was ensured by recording ¹H,¹H—COSY, —NOESY, ¹³C,¹H-HSQC and —HMBC experiments. The NMR of all prepared compounds are in accordance with NMR reported in the literature.

### Example 1

### Catalytic Oxidation of Carveol Using Complex [Zr(L)(Acac)2] or [Zr(L′)(Acac)₃] Acac=Acetylacetonate)

A mixture of Carveol (Aldrich ~1:1 isomers mixture), [Zr(L)(acac)₂] or [Zr(L′)(acac)₃] (1 mol. %) and cyclohexanone (1.5 eq.) was heated up to 130° C. in a glass reactor equipped with a stirrer, a thermometer, and a condenser. The reaction mixture was heated at 130° C. for 4 hours to complete conversion of carveol into carvone. The results with various L or L′ ligand taken from Table 2 are shown in Table 1.

TABLE 1
oxidation of carveol using complex [Zr(L)(acac)2]
or [Zr(L′)(acac)₃]

| | Conversion | Carvone | Selectivity |
|---|---|---|---|
| L | [GC %] | [GC %] | [GC %] |
| L1 | 76% | 72.3% | 95% |
| L2 | 79% | 74.2% | 94% |
| L′ | 81% | 76.1% | 94% |
| L3 | 98% | 97.1% | 99% |
| L4 | 98.2% | 97% | 99% |
| L5 | 97.7% | 96.9% | 99% |
| L6 | 95.1% | 88.1% | 93% |
| L7 | 98.8% | 98.0% | 99% |

TABLE 2
Structures of biphenolate or phenolate used

| Ligand | Structure^{a)} | Ligand | Structure^{a)} |
|---|---|---|---|
| L1 | 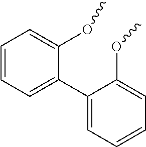 | L4 | 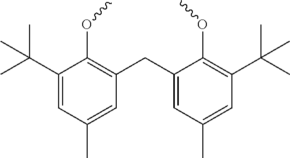 |
| L2 | 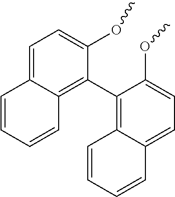 | L5 | 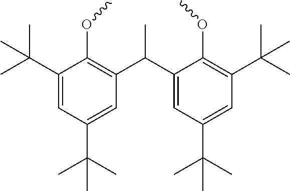 |
| L′ | 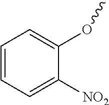 | L6 | 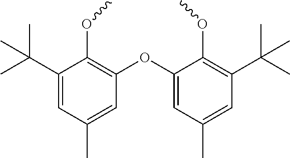 |
| L3 | 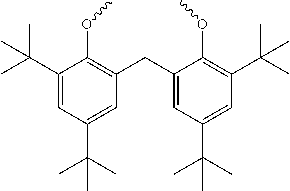 | L7 | 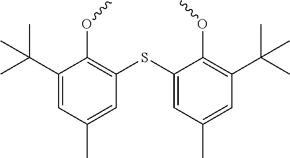 |

^{a)}The wavy line indicates the location of the bond between the Zirconium atom and L or L′

### Example 2

### Preparation of R—(−)-Carvone Via One-Pot Successive Rearrangement of R-(+)-1,2-limonene oxide Using Zn(L-Prolinate)₂ as a Catalyst and Oppenauer Oxidation of R—(−)-carveol in the Presence of Zirconium, [2,2′-methylenebis[(4-methyl-6-tert-butyl)phenolato]]bis(2,4-pentanedionato) and cyclohexanone as hydrogen Acceptor

A mixture of of R-(+)-1,2-limonene oxide (R-LMO), Zn(L-Prolinate)₂ (0.25 mol. %) and 2-aminophenol (4 eq./Zn) was heated at 185° C. for 9 hours in a glass reactor equipped with a stirrer, a thermometer, a Dean-Stark trap, and a condenser. When the rearrangement step was completed, the reaction mixture was cooled down to room temperature. 1 mol. % of Zirconium, [2,2′-methylenebis[(4-methyl-6-tert-butyl)phenolato]]bis(2,4-pentanedionato) and 1.1 equivalent of cyclohexanone compared to initial R-LMO were added. The reaction mixture was heated at 130° C. for 4 hours to complete conversion of carveol into carvone. The distillation afforded carvone in 99% yields based on intermediate carveol title and 82% based on initial R-LMO. This material was purified using conventional methods to obtain R—(−)-carvone in >99GC % purity.

### Example 3

### Preparation of R—(−)-carvone Via One-Pot Successive Rearrangement of R-(+)-1,2-limonene oxide Using Zn(L-Prolinate)₂ as a Catalyst and Oppenauer Oxidation of R—(−)-carveol in the Presence of Zirconium, [2,2′-methylenebis[(4-methyl-6-tert-butyl)phenolato]]bis(2,4-pentanedionato) and benzaldehyde as hydrogen Acceptor

A mixture of of R-(+)-1,2-limonene oxide (R-LMO), Zn(L-Prolinate)₂ (0.25 mol. %) and 2-aminophenol (4 eq./Zn) was heated at 185° C. for 9 hours in a glass reactor equipped with a stirrer, a thermometer, a Dean-Stark trap, and a condenser. When the rearrangement step was completed, the reaction mixture was cooled down to room temperature. 1 mol. % of Zirconium, [2,2′-methylenebis[(4-methyl-6-tert-butyl)phenolato]]bis(2,4-pentanedionato) and 1.1 equivalent of benzaldehyde compared to initial R-LMO were added. The reaction mixture was heated at 130° C. for 4 hours to complete conversion of carveol into carvone. The distillation afforded carvone in 96% yields based on intermediate carveol title and 79% based on initial R-LMO. This material was purified using conventional methods to obtain R—(−)-carvone in >99GC % purity.

### Example 4

### Preparation of R—(−)-carvone Via One-Pot Successive Rearrangement of R-(+)-1,2-limonene oxide Using Zn(L-Prolinate)₂ as a Catalyst and Oppenauer Oxidation of R—(−)-carveol in the Presence of Zirconium, [2,2′-methylenebis[(4-methyl-6-tert-butyl)phenolato]]bis(2,4-pentanedionato) and benzaldehyde as hydrogen Acceptor

A mixture of of R-(+)-1,2-limonene oxide (R-LMO), Zn(L-Prolinate)₂ (0.25 mol. %) and 2-aminophenol (4 eq./Zn) was heated at 185° C. for 9 hours in a glass reactor equipped with a stirrer, a thermometer, a Dean-Stark trap, and a condenser. When the rearrangement step was completed, the reaction mixture was cooled down to room temperature. 1 mol. % of Zirconium, [2,2′-methylenebis[(4-methyl-6-tert-butyl)phenolato]]bis(2,4-pentanedionato) and 1.1 equivalent of benzaldehyde compared to initial R-LMO were added. The reaction mixture was heated at 100° C. for 2 hours then 130° C. for further 2 hours to complete conversion of carveol into carvone. The distillation afforded carvone in 99% yields based on intermediate carveol title and 82% based on initial R-LMO. This material was purified using conventional methods to obtain R—(−)-carvone in >99GC %.

### Example 5

### Preparation of R—(−)-carvone Via One-Pot Successive Rearrangement of R-(+)-1,2-limonene oxide Using Zn(2-ethylhexanoate)₂ as a Catalyst and Oppenauer Oxidation of R—(−)-carveol in the Presence of Zirconium, [2,2′-methylenebis[(4-methyl-6-tert-butyl)phenolato]]bis(2,4-pentanedionato) and benzaldehyde as hydrogen Acceptor

A mixture of of R-(+)-1,2-limonene oxide (R-LMO), Zn(2-Ethylhexanoate)₂ (0.5 mol. %) and 2-aminophenol (3 eq./Zn) was heated at 185° C. for 15 hours in a glass reactor equipped with a stirrer, a thermometer, a Dean-Stark trap, and a condenser. When the rearrangement step was completed, the reaction mixture was cooled down to room temperature. 1 mol. % of Zirconium, [2,2′-methylenebis[(4-methyl-6-tert-butyl)phenolato]]bis(2,4-pentanedionato) and 1.1 equivalent of benzaldehyde compared to initial R-LMO were added. The reaction mixture was heated at 100° C. for 2 hours then 130° C. for further 2 hours to complete conversion of carveol into carvone. The distillation afforded carvone in 99% yields based on intermediate carveol title and 83% based on initial R-LMO. This material was purified using conventional methods to obtain R—(−)-carvone in >99GC % purity.

### Example 6

### Preparation of R—(−)-carvone Via One-Pot Successive Rearrangement of R-(+)-1,2-limonene oxide Using Zn(2-ethylhexanoate)₂ as a Catalyst and Oppenauer Oxidation of R—(−)-carveol in the Presence of Zirconium, [2,2′-methylenebis[(4-methyl-6-tert-butyl)phenolato]]bis(2,4-pentanedionato) Generated In Situ and Benzaldehyde as hydrogen Acceptor

A mixture of of R-(+)-1,2-limonene oxide (R-LMO), Zn(2-Ethylhexanoate)₂ (0.5 mol. %) and 2-aminophenol (3 eq./Zn) was heated at 185° C. for 15 hours in a glass reactor equipped with a stirrer, a thermometer, a Dean-Stark trap, and a condenser. When the rearrangement step was completed, the reaction mixture was cooled down to 30° C. 1 mol. % of Zr(OPr)₄ (70% in propanol), 6,6′-methylenebis(2-(tert-butyl)-4-methylphenol) and 2.2 eq. of acetylacetone were added and the reaction mixture was stirred for 1 h. 1.1 equivalent of benzaldehyde compared to initial R-LMO were then added. The reaction mixture was heated at 100° C. for 2 hours then 130° C. for further 2 hours to complete conversion of carveol into carvone. The distillation afforded carvone in 99% yields based on intermediate carveol title and 83% based on initial R-LMO. This material was purified using conventional methods to obtain R—(−)-carvone in >99GC % purity.

### Example 7

### Catalytic Oxidation of (6,6-dimethylbicyclo[3.1.1]hept-2-en-2-yl)methanol Using the Invention Conditions

A mixture of (6,6-dimethylbicyclo[3.1.1]hept-2-en-2-yl)methanol (10 g, 65.7 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 79% conversion. After distillation on residues the expected product i.e. 6,6-dimethylbicyclo[3.1.1]hept-2-ene-2-carbaldehyde was obtained in 91% yield.

### Example 8

### Catalytic Oxidation of (E)-4-methyldec-3-en-5-ol Using the Invention Conditions

A mixture of (E)-4-methyldec-3-en-5-ol (5 g, 29.4 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 92% conversion. After distillation on residues the corresponding ketone i.e. (E)-4-methyldec-3-en-5-one was obtained in 98% yield.

### Example 9

### Catalytic Oxidation of 6,6-dimethyl-2-methylenebicyclo[3.1.1]heptan-3-ol Using the Invention Conditions

A mixture of 6,6-dimethyl-2-methylidenebicyclo[3.1.1]heptan-3-ol 2 g, 13.1 mmol Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 82% conversion. After distillation on residues 6,6-dimethyl-2-methylenebicyclo[3.1.1]heptan-3-one was obtained in 910% yield.

### Example 10

### Catalytic Oxidation of (4-(prop-1-en-2-yl)cyclohex-1-en-1-yl)methanol Using the Invention Conditions

A mixture of (4-(prop-1-en-2-yl)cyclohex-1-en-1-yl)methanol (4 g, 25.9 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 80% conversion. After distillation on residues, 4-(prop-1-en-2-yl)cyclohex-1-ene-1-carbaldehyde was obtained in 95% yield.

### Example 11

### Catalytic Oxidation of 1-octen-3-ol Using the Invention Conditions

A mixture of 1-octen-3-ol (4 g, 31.2 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 38% conversion. After distillation on residues 1-octen-3-one was obtained in 97% yield.

### Example 12

### Catalytic Oxidation of 4,7,7-trimethylbicyclo[4.1.0]hept-4-en-3-ol Using the Invention Conditions

A mixture of 4,7,7-trimethylbicyclo[4.1.0]hept-4-en-3-ol (2 g, 13.1 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 82% conversion and 70% selectivity on 7,7-trimethylbicyclo[4.1.0]hept-4-en-3-one.

### Example 13

### Catalytic Oxidation of Geraniol Using the Invention Conditions

A mixture of geraniol (4 g, 25.9 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 68% conversion and 87% selectivity on (2E)-3,7-dimethylocta-2,6-dienal.

### Example 14

### Catalytic Oxidation of Nerol Using the Invention Conditions

A mixture of nerol (4 g, 25.9 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 58% conversion and 85% selectivity on (2Z)-3,7-dimethylocta-2,6-dienal.

### Example 15

### Catalytic Oxidation of 4,6,6-trimethylbicyclo[3.1.1]hept-3-en-2-ol Using the Invention Conditions

A mixture of 4,6,6-trimethylbicyclo[3.1.1]hept-3-en-2-ol (5 g, 32.8 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 99% conversion and 98% selectivity on 4,6,6-trimethylbicyclo[3.1.1]hept-3-en-2-one.

### Example 16

### Catalytic Oxidation of (2E,6Z)-nona-2,6-dien-1-ol Using the Invention Conditions

A mixture of (2E,6Z)-nona-2,6-dien-1-ol (4 g, 28.5 mmol), Zirconium, [2,2′-methylenebis[(4-methyl-6-tert-butyl)phenolato]]bis(2,4-pentanedionato) (1 mol. %) and benzaldehyde (1.1 eq.) was heated at 130° C. for 2 hours in a Schlenk equipped with a magnetic stirrer, a thermometer and a condenser to 65% conversion and 90% selectivity on (2E,6Z)-nona-2,6-dienal.

### Example 17

### Catalytic Oxidation of 3-methyl-2-buten-1-ol Using the Invention Conditions

A mixture of 3-methyl-2-buten-1-ol i.e. prenol (4 g, 46.4 mmol), Zirconium, [2,2′-methylenebis[(4-methyl-6-tert-butyl)phenolato]]bis(2,4-pentanedionato)_(1 mol. %) and benzaldehyde (1.1 eq.) was heated at 130° C. for 2 hours in a Schlenk equipped with a magnetic stirrer, a thermometer and a condenser to 45% conversion and 95% selectivity on 3-methyl-2-butenal i.e prenal.

### Example 18

### Preparation of R—(−)-carvone Via One-Pot Successive Rearrangement of R-(+)-1,2-limonene oxide and Oppenauer Oxidation of R—(−)-carveol in Invention's Conditions and Comparative Conditions

A mixture of 1,2-limonene oxide (60% cis isomer and 40% trans isomer, 400 g), the catalyst and 2-amino phenol (2-AP) (equivalents of 2-AP related to Zn are specified in Table 3), was heated up to the temperature specified in Table 3, in a glass reactor equipped with a stirrer, a thermometer, a Dean-Stark trap, and a condenser. The temperature was maintained till conversion of R-LMO was >96%. After cooling down the oxidation catalyst was added together with the hydrogen acceptor (amount is specified in Table 3). In some examples the hydrogen acceptor is added portion wise after selective removal of the hydrogen acceptor (HA) excess and its corresponding alcohol. To clarify, if 2/2 is reported on the time column, this means that half of the HA total amount was added first and after 2 h heating at the reported temperature, the non-reacted HA together with its corresponding alcohol were removed by distillation, then the operation is repeated; e.g. 2/2/2 means that HA was added in 3 times every 2 hours. Crude Carvone was then purified by distillations to obtain Carvone >98% purity. Yield is reported in the column Carvone isolated yield. Separately or together the HA excess and its corresponding alcohol are distilled too. Their overall yield ((mol HA excess+ mol Alcohol)/mol initial total HA) is also reported when possible.

TABLE 3
Rearrangement of 1,2-limonene oxide into carveol followed by oxide and Oppenauer oxidation
to obtained R-(−)-carvone in one-pot using invention's conditions and comparative conditions

| | | Catalyst | 2-AP | T | Carveol | Carvone | | Catalyst |
|---|---|---|---|---|---|---|---|---|
| Entry | Catalyst | mol. % | (eq/Zn) | (° C.) | GC % | GC % | Catalyst | mol. % |
| 1¹⁾ | Zn | 1 | 1.5 | 202- | 70% | 13% | Zn | 3 |
| | (octoate)₂ | | | 227 | | | (octoate)₂ | |
| 2²⁾ | Zn | 0.65 | 5.8 | 202- | 74.5% | 4% | Zn | 0.65 |
| | (stearate)₂ | | | 227 | | | (stearate)₂ | |
| 3²⁾ | Zn | 0.65 | 5.8 | 202- | 70.2% | 7.3% | Zn | 0.65 |
| | (Naphtenate)₂ | | | 227 | | | (Naphtenate)₂ | |
| 4¹⁾ | Zn | 1 | 1.5 | 202- | 71.7% | 13% | Zn | 3 |
| | (octoate)₂ | | | 227 | | | (octoate)₂ | |
| 5³⁾ | Zn | 1 | 1.5 | 202- | 70.4% | 12.7% | Zn | 3 |
| | (octoate)₂ | | | 227 | | | (octoate)₂ | |
| 6⁴⁾ | Zn | 0.25 | 4 | 185 | 83.5% | 5.8% | [RaloxZr | 1 |
| | (octoate)₂ | | | | | | (acac)₂]⁵⁾ | |
| 7⁴⁾ | Zn | 0.25 | 4 | 185 | 83.2% | 5.4% | [RaloxZr | 1 |
| | (octoate)₂ | | | | | | (acac)₂]⁵⁾ | |
| 8⁴⁾ | Zn | 0.25 | 4 | 185 | 84.4% | 4.7% | [RaloxZr | 1 |
| | (proline)₂ | | | | | | (acac)₂]⁵⁾ | |
| | | | | | | | | HA |
| | | | | | | | | exces + |
| | Hydrogen | | | | | | Carvone | alcohol |
| | acceptor | HA | Time | T | Carveol | Carvone | Isolated | isolated |
| Entry | (HA) | eq/LO⁶⁾ | (h) | (° C.) | GC % | GC % | yield | yield |
| 1¹⁾ | Cyclohexanone | 1.5 | 2/2/2 | 190 | 0.9% | 82.0% | 70.0% | 83% |
| 2²⁾ | Cyclohexanone | 1.8 | 2/2 | 190 | 38.4% | 43.8% | | disposed |
| 3²⁾ | Cyclohexanone | 1 | 4 | 190 | 18.1% | 56.1% | | disposed |
| 4¹⁾ | Benzaldehyde | 1.5 | 2/2/2 | 190 | 5.0% | 74.0% | 65.0% | 71% |
| 5³⁾ | Benzaldehyde | 1.5 | 2/2/2 | 130 | 51.5% | 24.1% | | |
| 6⁴⁾ | Cyclohexanone | 1.1 | 2 | 130 | | | 82.0% | 90% |
| 7⁴⁾ | Benzaldehyde | 1.1 | 4 | 100- | 0.4% | 89.0% | 83.0% | 95% |
| | | | | 130 | | | | |
| 8⁴⁾ | Benzaldehyde | 1.1 | 4 | 100- | 0.0% | 91.0% | 82.0% | 97% |
| | | | | 130 | | | | |

¹⁾ Conditions as reported in WO2003004448
²⁾ Conditions as reported in WO2021151790
³⁾ Conditions as reported in WO2003004448; ecept the temperature of the oxidation
⁴⁾ Invention's conditions
⁵⁾ 2,2′-methylenebis[(4-methyl-6-tert-butyl)phenol]
⁶⁾ 1,2-limonene oxide

The invention's conditions, contrary to the prior art conditions, allow isolating carvone in high yield while recovering the hydrogen acceptor or the corresponding alcohol very efficiency. When the hydrogen acceptor is benzaldehyde, the reduced produced formed is the benzylic alcohol which is a valuable ingredient which could be used as a perfuming ingredient.

1. A process for the oxidation of an allylic alcohol into an alpha,beta-unsaturated carbonyl compound, wherein said process is carried out in the presence of
i) a catalyst of formula 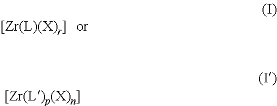
wherein L is a bisphenolate, a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units; and
ii) a hydrogen acceptor.

2. The process according to claim 1, wherein the phenolate is of formula 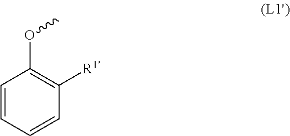
wherein the wavy line indicates the location of the bond between the Zirconium atom and L; R^{1′} is a hydrogen atom, a halogen atom, a cyano group, a nitro group, a C₁₋₆ alkoxy group, a C₁₋₆ alkyl group optionally substituted with one or more halogen atoms or a COOR group wherein R is a hydrogen atom or a C₁₋₆ alkyl group.

3. The process according to claim 2, wherein the phenolate is selected from the group consisting of 2-nitrophenolate, ortho-cresolate, 2-(trifluoromethyl)phenolate and 2-cyanophenolate.

4. The process according to claim 1, wherein the bisphenolate is of formula 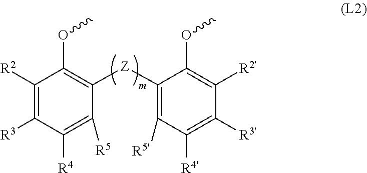
wherein the wavy lines indicate the location of the bond between the Zirconium atom and L; m is 0 or 1; R², R³ R⁴, R⁵, R^{2′}, R^{3′}, R^{4′}, and R^{5′}, when taken separately, independently from each other, are a hydrogen, a halogen atom, a nitro group, a 2H-benzo[d][1,2,3]triazol-2-yl group, a C₁₋₆ alkoxy group, a C₁₋₆ thioalkyl group or a C₁₋₁₀ alkyl group optionally substituted with one or more of a halogen atom, hydroxy, amine or C₁₋₃ alkoxy group; or R² and R³ or R³ and R⁴ or R^{2′} and R^{3′} or R^{3′} and R^{4′}, when taken together represent a —O—(CH₂)_{y}—O— group wherein y is 1 or 2 or form a C₆ aryl, C₅₋₆ cycloalkyl group, each optionally substituted with one or more of a halogen atom, hydroxy or C₁₋₃ alkoxy group; and Z is an oxygen or sulphur atom, a (—CH₂—)₂ group, a —NH— group, a —SO₂— group, a —S—S— group, a —CH₂—NH—CH₂— group, or a —C(R⁶)(R⁷)— group wherein R⁶ and R⁷, when taken separately, independently from each other, are a hydrogen atom or a C₆ aryl, C₆ heteroaryl or a C₁₋₃ alkyl group, each optionally substituted by one to three halogen atoms or C₁₋₃ alkoxy groups, and wherein the heteroatom is one or more of an oxygen or nitrogen atom.

5. The process according to claim 4, wherein the bisphenolate is selected from the group consisting of [1,1′-biphenyl]-2,2′-diol, [1,1′-binaphthalene]-2,2′-diol, 6,6′-methylenebis(2,4-di-tert-butylphenol), 6,6′-(ethane-1,1-diyl)bis(2,4-di-tert-butylphenol), 6,6′-methylenebis(2-(tert-butyl)-4-methylphenol), 6,6′-oxybis(2-(tert-butyl)-4-methylphenol) and 6,6′-thiobis(2-(tert-butyl)-4-methylphenol).

6. The process according to claim 1, wherein the anionic ligand, independently from each other, is a halogen atom, a β-diketonate, a OOCR⁸ group or a OR⁹ group wherein R⁸ is a C₁₋₁₀ alkyl group, a benzyl group, a naphtyl group or a phenyl group optionally substituted by a hydroxy group and R⁹ is a C₁₋₆ alkyl group.

7. The process according to claim 1, wherein the hydrogen acceptor is a hydrocarbon comprising at least one carbonyl functional group and having a boiling point equal or greater than 80° C.

8. The process according to claim 1, wherein the hydrogen acceptor is selected from the group consisting of benzaldehyde, cyclohexanone, octalynone, 2-heptanone, 2-octanone, 2-pentanone, acetophenone, 4-methyl-2-pentanone, 3-methyl-2-butanone, isophorone and a mixture thereof.

9. The process according to claim 1, wherein the allylic alcohol is carveol and the alpha,beta-unsaturated carbonyl compound is carvone.

10. The process according to claim 1, wherein the process comprises the step of
i) the rearrangement of an epoxide into an allylic alcohol; and
ii) the oxidation of the allylic alcohol obtained in step i) into the alpha,beta-unsaturated carbonyl compound.

11. The process according to claim 10, wherein the rearrangement of the epoxide into the allylic alcohol is carried out in the presence of a catalyst of formula Zn(aa)₂ wherein aa is an α-amino carboxylate having at least 3 carbon atoms or a β-amino carboxylate; and an amino phenol.

12. The process according to claim 11, wherein the α-amino acid is selected from the group consisting of proline, valine, lysine, D-proline, D-valine, D-lysine L-proline, L-valine, L-lysine, 2-phenylglycine, phenyl alanine, 2-amino-2-methylpropanoic acid and a mixture thereof and the amino phenol is selected from the group consisting of 2-aminophenol, 2-aminomethylphenol and a mixture thereof.

13. The process according to claim 10, wherein the rearrangement of the epoxide into the allylic alcohol is carried out in the presence of a catalyst of formula Zn(carboxylate)₂ and an amino phenol.

14. The process according to claim 10, wherein the epoxide is 1,2-limonene oxide.

15. A catalytic system comprising or consisting of
i) a catalyst of formula 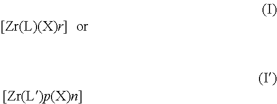
wherein L is a bisphenolate, a triphenolate or a calixarene with at least 4 phenol units, L′ is a phenolate, X is an anionic ligand, p is 1 when n is 3 or p is 2 when n is 2 or p is 0 when n is 4 and r is 2 when L is a bisphenolate or r is 1 when L is a triphenolate or r is 0 when L is a calixarene with at least 4 phenol units; and
ii) a hydrogen acceptor.

16. The process according to claim 7, wherein the hydrogen acceptor is a hydrocarbon comprising at least one carbonyl functional group and having a boiling point greater than 120° C.
